(19) United States
(12) Patent Application Publication
(10) Pub. No.: US 2011/0137046 A1
(43) Pub. Date: Jun. 9, 2011

(54) PREPARATION METHOD OF (4S,5R)-SEMIESTER

(76) Inventors: Fen-Er Chen, Shanghai (CN); Fei Xiong, Shanghai (CN); Xu-Xiang Chen, Shanghai (CN); Lei Zhao, Shanghai (CN); Zhong-Hua Wang, Shanghai (CN)

(21) Appl. No.: 12/996,078

(22) PCT Filed: Jun. 5, 2009
(86) PCT No.: PCT/CN09/00627

§ 371 Date: Feb. 18, 2011

(30) Foreign Application Priority Data
Jun. 5, 2008 (CN) 200810038584.6
Sep. 4, 2008 (CN) 200810042506.3

(51) Int. Cl. C07D 233/02

(52) U.S. Cl. 548/322.5

(57) ABSTRACT

A preparation method of (4S,5R)-semiester in which cycloanhydride conducts enantioselective ring-opening with alcohol in the presence of 9-epiquininurea. With this method, (4S,5R)-semiester is prepared at room temperature with high yield and high stereoselectivity.

### TECHNICAL FIELD

The present invention belongs to the field of organic chemistry, and is related to the preparation method of (4S, 5R)-semiester by the use of 9-epiquininurea

### TECHNICAL BACKGROUND

(45, 5R)-semiester represented by general formular (I) is the key intermediate to synthesize (+)-biotin (vitamin Currently, the preparation of the compound includes chiral resolution method, chiral auxiliary method and asymmetric catalysis method. The resolution method was first reported by Gerecke et al. (Helv Chim Acta, 1970, 53, 991) for preparation of racemic CAC monocyclohexanol ester via monoesterification between cycloanhydride (II) and cyclohexanol, then conduct direct enantiomorphous crystallization with pseudoephedrine and resolve to get desired (4S, 5R)-semiester (I). German patent 2058234, Chinese patent 106365, European patent 92194 and Chen Fen-Er et al. (Chemical Journal of Chinese Universities, 2001, 12, 1141) respectively reported preparation of (4S, 5R)-semiester represented by general formular (I) using dehydroabietylamine, substituted chiral diphenyl ethamine and chloromycetin by product (15,25)-threo-1-(p-nitrophenyl)-1,3-propanediol as resolution agent. But those resolution methods have disadvantages of high price, insufficient raw material resource, poor resolution efficiency and uneasy recovery.

Gerecke et al. (Helv Chim Acta, 1970, 53, 991) reported formation of diastereoisomer CAC semi-ester ex cycloanhydride (II) with cholesterin as chiral auxiliary, then via recrystallization and isolation to give (4S, 5R)-semiester (I). European patent 92194 used optically active substituted chiral secondary alcohol and tert-butyl alcohol as chiral auxiliary to prepare (4S, 5R)-semiester (I). But the chiral auxiliaries used in the above-mentioned methods have disadvantages of high price, difficult preparation and uneasy recovery.

European patent 84892, Chen Fen-Er et al. (Advanced Synthesis & Catalysis, 2005, 347, 549) respectively reported the preparation of (4S, 5R)-semiester represented by general formular (I) via stereoselective hydrolysis of meso-diester using pig liver esterase and poly pig liver esterase as catalyst. Chinese patent 1473832, 101157655 respectively described the preparation of (4S, 5R)-semiester represented by general formular (I) via asymmetric alcoholysis of cycloanhydride (II) using chiral amine (1S, 2S)-1-(4-nitrophenyl)-2-N,N-dimethylamino-3-triphenylmethoxy-1-propanol and 9-propargylquinine as catalyst. But those methods have weakness of small production scale, complex operation and strict reaction temperature.

### SUMMARY OF INVENTION

The aim of the said invention is to overcome the disadvantages of existing technology and provide a preparation method of (4S, 5R)-semiester represented by general formular (I) with moderate conditions, high yield and high stereoselectivity.

The said invention conducts enantioselective ring-opening between cycloanhydride (II) and alcohol with presence of 9-epiquininurea to prepare (4S, 5R)-semiester represented by general formular (I) with yield >95% and e.e.>98%. The synthetic route is as follows:

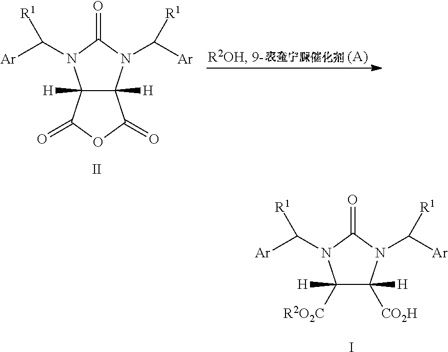

Where in the formula R¹ is hydrogen, C₁˜C₆ alkyl, phenyl, alkyl substituted phenyl or alkoxyl substituted phenyl, Ar is phenyl, alkyl substituted phenyl or alkoxyl substituted phenyl, nitro-substituted phenyl, phenyl halide, thienyl, furyl or naphthyl; R² is C₁˜C₆ alkyl, C₃˜C₆ naphthene, C₂˜C₆ alkenyl, aralkyl or aralkenyl.

In the said asymmetric monoesterification, catalyst 9-epiquininurea has structure as indicated in Formular A. It enables the performance of reaction at room temperature and preparation of (4S, 5R)-semiester represented by general formular (I) with high yield and high stereoselectivity. Besides, the said chiral catalyst has convenient synthesis, wide availability of raw materials, and can be quantitatively recovered, which is liable for industrialized production.

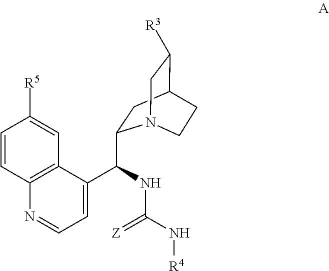

Where R³ is hydrogen, C₁˜C₆ alkyl, C₂˜C₆ alkenyl or C₂˜C₆ alkynyl; R⁴ is hydrogen, C₁˜C₆ alkyl, C₂˜C₆ alkenyl, C₂˜C₆ alkynyl, C₃˜C₆ naphthene, aryl or substituted derivative of any above-mentioned group; R⁵ is —H or —OR⁶, R⁶ is C₁˜C₆ alkyl, C₃˜C₆ naphthene, C₂˜C₆ alkenyl, C₂˜C₆ acyl, benzyl, benzoyl, cinnamyl or substituted derivative of any above-mentioned group; Z is O, S or Se.

The alcohol used is C₁˜C₆ alkanol, C₃˜C₆ naphthenic alcohol, C₂˜C₆ enol, aralkyl alcohol, arenol or substituted derivative of any above-mentioned alcohol, such as methanol, ethanol, n-propanol, isopropanol, n-butanol, isobutanol, cyclohexanol, ally' alcohol, benzyl alcohol, cinnamyl alcohol etc. for asymmetric monoesterification. Those alcohols are cheap and easily available. The used organic solvent includes halohydrocarbon (e.g. dichloro-methane, chloroform, 1,2-dichloroethane, carbon tetrachloride etc.); aliphatic hydrocarbon (e.g. hexane, heptane, octane, nonane, acetonitrile, ethyl acetate etc.); arene (e.g. benzene, toluene, xylene, nitrobenzene etc.); various haloarene (e.g. chlorobenzene etc.) or ether (e.g. diethyl ether, MTBE, THF or 1,4-dioxane etc.). These solvents are widely available, cheap and easy for recovery. When mol ratio among cycloanhydride (II)/alcohol/chiral catalyst is at 1:1˜10:0.01˜2.2, reaction can be smoothly completed. Control reaction temperature at −15° C.˜50° C., reaction time at 4˜80 hrs for reaction completion.

In the said invention, the preferred chiral catalyst is 9-epiquininurea (A) with vinyl as R³; —OR⁵ as R⁴, methyl as R⁵, S atom as Z. The catalyst has advantages of convenient synthesis, wide raw material resource and easy recovery.

In the said invention, the alcohol used is methanol, which is widely available with low price.

In the said invention, the mol ratio among cycloanhydride (II)/alcohol/chiral catalyst is preferred at 1:3˜10:0.01˜1.1.

In the said invention, the preferred reaction temperature is at 0˜25° C.

In the said invention, the preferred reaction time is at 10˜36 hrs.

In the said invention, the preferred organic solvent is MTBE, which is environmental friendly, widely available with low price.

The said invention has moderate reaction conditions, easy operation, and cheap raw materials with easy availability. What's more, the obtained product has high yield and high stereoselectivity and the catalyst can be quantitatively recovered and recycled. So, the catalyst has low cost and is suitable for industrialized production.

### EXAMPLES

The following examples can better describe the content of the said invention. But the said invention is not limited by those examples.

### Example 1

In a dry flask, transfer cis-1,3-dibenzylimidazoline-2-one-2H-furan[3,4-d]imidazole-2,4,6-trione (33.6 g, 0.10 mol), catalyst A (R³═—CH═CH₂, R⁴═—OR⁵, R⁵═CH₃, Z═S) (65.34 g, 0.11 mol), MTBE (4L), drop anhydrous methanol (40.4 mL, 1 mol) at 25° C., then continuously stir for 24 hrs. Upon reaction, add 2M hydrochloric acid (400 mL) into remnant, stir for 10 mins, stay still, separate out organic layer and dry with anhydrous sodium sulfate. Filter, recover solvent ex filtrate under vacuum to get white crystal powder I (R¹═—H, Ar═—Ph, R²═—CH₃, 36 g, 98%) with m.p. 149˜150° C., [α]_{D}²²=+2.74° (c 0.20, CHCl₃).

IR (KBr): v=2979, 2384, 2281, 1742, 1463, 1229, 1169, 767cm⁻¹.

¹H NMR (CDCl₃): δ=3.54 (s, 1H, OCH₃), 4.00˜4.04 (m, 2H, C₆ₐ—H, C₃ₐ—H), 4.16˜4.80 (dddd, 4H, 2*CH₂C₆H₅), 7.19˜7.53 (m, 10H, 2*ArH) ppm.

EI-MS: (m/z, %)=368 (M⁺, 37), 323 (46), 309 (59), 265 (44), 154 (8), 136 (18), 91 (100).

Catalyst recovery: adjust the separated aqueous layer of hydrochloric acid with 20% NaOH solution to pH 14. Filter the isolated white solid, dry to quantitatively recover catalyst.

### Example 2

In a dry flask, transfer cis-1,3-dibenzylimidazoline-2-one-2H-furan[3,4-d]imidazole-2,4,6-trione (33.6 g, 0.10 mol), catalyst A (R³═—CH═CH₂, R⁴═—OR⁵, R⁵═CH₃, Z═S) (5.94 g, 0.01 mol), 1,4-dioxane (8L), drop propiolic alcohol (58.2 mL, 1 mol) at 25° C., then continuously stir for 24 hrs. Upon reaction, add 2M hydrochloric acid (40 mL) into remnant, stir for 10 mins, stay still, separate out organic layer and dry with anhydrous sodium sulfate. Filter, recover solvent ex filtrate under vacuum to get white crystal powder I (R¹═—H, Ar═—Ph, R²═propargyl, 37.2 g, 95%) with m.p. 132.7˜135.8° C., [α]_{D}²⁵=+14.3° (c 1.0, CHCl₃).

### Example 3

In a dry flask, transfer cis-1,3-dibenzylimidazoline-2-one-2H-furan[3,4-d]imidazole-2,4,6-trione (33.6 g, 0.10 mol), catalyst A (R³═—CH═CH₂, R⁴═—OR⁵, R⁵═CH₃, Z═S) (65.34 g, 0.11 mol), 1,4-dioxane (4L), drop anhydrous methanol (40.4 mL, 1.0 mol) at 25° C., then continuously stir for 24 hrs. Upon reaction, add 2M hydrochloric acid (400 mL) into remnant, stir for 10 mins, stay still, separate out organic layer and dry with anhydrous sodium sulfate. Filter, recover solvent ex filtrate under vacuum to get white crystal powder I (R¹═—H, Ar═—Ph, R²═—CH₃, 35.2 g, 96%) with m.p. 148˜150° C., [α]_{D}²²=+2.70° (c 0.20, CHCl₃).

### Example 4

In a dry flask, transfer cis-1,3-dibenzylimidazoline-2-one-2H-furan[3,4-d]imidazole-2,4,6-trione (33.6 g, 0.10 mol), catalyst A (R³═—CH₂CH₃, R⁴═—OR⁵, R⁵═CH₃, Z═S) (65.34 g, 0.11 mol), THF (4L), drop anhydrous methanol (40.4 mL, 1.0 mol) at 25° C., then continuously stir for 24 hrs. Upon reaction, add 2M hydrochloric acid (400 mL) into remnant, stir for 10 mins, stay still, separate out organic layer and dry with anhydrous sodium sulfate. Filter, recover solvent ex filtrate under vacuum to get white crystal powder I (R¹═—H, R²═—CH₃, 35 g, 95%) with m.p. 147˜150° C., [a]_{D}²²=+2.70° (c 0.20, CHCl₃).

1. A preparation method of (4S, 5R)-semiester (I), 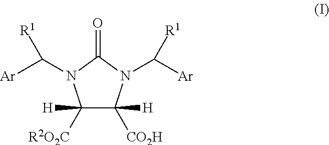
Characterized in that cycloanhydride (II) conducts enantioselective ring-opening reaction in the presence of 9-epiquininurea to prepare (4S, 5R)-semiester (I): 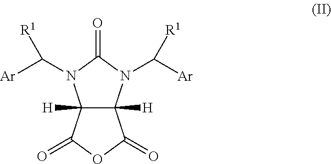
R¹ is hydrogen, C₁˜C₆ alkyl, phenyl, p-tolyl, p-methoxyphenyl, 3,4-dimethylphenyl, 3,4-dimethoxyphenyl, 3,4,5-trimethylphenyl, 3,4,5-trimethoxyphenyl, p-chlorophenyl, Ar is phenyl, p-methoxyphenyl, 3,4-dimethylphenyl, 3,4-dimethoxyphenyl, 3,4,5-trimethylphenyl, 3,4,5-trimethoxyphenyl, p-chlorophenyl, thienylphenyl, furyl or naphthyl; R² is C₁˜C₆ alkyl, C₃˜C₆ naphthene, C₂˜C₆ alkenyl, aralkyl or aralkenyl,
Wherein said 9-epiquininurea has structure A as indicated: 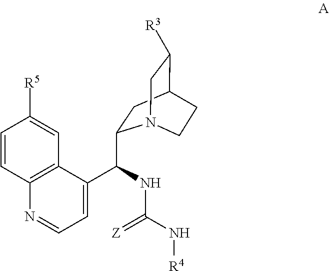
Where R³ is hydrogen, C₁˜C₆ alkyl, C₂˜C₆ alkenyl or C₂˜C₆ alkynyl; R⁴ is hydrogen, C₁˜C₆ alkyl, C₂˜C₆ alkenyl, C₂˜C₆ alkynyl, C₃˜C₆ naphthene, aryl or substituted derivative of any above-mentioned group; R⁵ is —H or —OR⁸, R⁶ is C₁˜C₆ alkyl, C₃˜C₆ naphthene, C₂˜C₆ alkenyl, C₂˜C₆ acyl, benzyl, benzoyl, cinnamyl or substituted derivative of any above-mentioned group; Z is O, S or Se.

2. The said method as described in claim 1, characterized in that said ring-opening reaction is carried out in the presence of alcohol.

3. The said method as described in claim 2, characterized in that said alcohol is C₁˜C₆ alkanol, C₃˜C₆ naphthenic alcohol, C₂˜C₆ enol, aralkyl alcohol, arenol or substituted derivative of any above-mentioned alcohol, preferably methanol, allyl alcohol, cyclohexanol, benzyl alcohol or cinnamyl alcohol.

4. The said method as described in claim 1, characterized in that the reaction is carried out with mol ratio among cycloanhydride (II)/alcohol/chiral catalyst is 1:1˜10:0.01˜2.2.

5. The said method as described in claim 1, characterized in that the reaction is carried out at a temperature of −15° C.˜50° C.

6. The said method as described in claim 1, characterized in that the reaction is carried out with the reaction time of 4˜80 hrs.

7. The said method as described in claim 1, characterized in that said reaction is carried out in organic solvent at room temperature, normal pressure, pressurerization or pressure reduction.

8. The said method as described in claim 7, characterized in that the said organic solvent is one or several of halohydrocarbon, aliphatic hydrocarbon, arene or ether.

9. The said method as described in claim 8, characterized in that said organic solvent is diethyl ether, MTBE, THF and/or 1,4-dioxane.

10. The said method as described in claim 1, characterized in that the mol ratio of cycloanhydride (II)/alcohol/chiral catalyst is at 1:3˜10:0.01˜1.1.

11. The said method as described in claim 1, characterized in that the reaction temperature is at 0° C.˜25° C.

12. The said method as described in claim 1, characterized in that the reaction time is 10˜72 hrs.

13. The said method as described in claim 1, characterized in that R³ is ethyl, vinyl, R⁴ is naphthene, aryl and their derivatives, R⁵ is —OR⁸, R⁶ is methyl and Z is S atom.
